(19) United States
(12) Patent Application Publication
(10) Pub. No.: US 2022/0369685 A1
(43) Pub. Date: Nov. 24, 2022

(54) COMPOSITIONS AND METHODS FOR THE IMPROVEMENT AND MAINTENANCE OF GLUCOSE METABOLISM IN CHILDHOOD AND ADOLESCENCE

(71) Applicant: SOCIETE DES PRODUITS NESTLE S.A., Vevey (CH)

(72) Inventors: FRANCOIS-PIERRE MARTIN, Vuisternens-devant-Romont (CH); JONATHAN PINKNEY, Plymouth (GB); JOANNE HOSKING, Plymouth (GB)

(21) Appl. No.: 17/759,385

(22) PCT Filed: Feb. 1, 2021
(86) PCT No.: PCT/EP2021/052264

§ 371 Date: Jul. 25, 2022

(30) Foreign Application Priority Data
Feb. 3, 2020 (EP) 20155174.4

(51) Int. Cl. A23L 33/175

(52) U.S. Cl. CPC A23L 33/175

(57) ABSTRACT

The present invention provides a nutritional composition for use in glucose management. The nutritional composition comprises one or more amino acids, or combinations thereof, selected from the group consisting of: hydroxyproline, proline, ornithine and alpha-aminobutyric acid, that can be particularly beneficial for children or adolescents for use in glucose management.

### FIELD OF INVENTION

The nutritional composition comprises one or more amino acids, or combinations thereof, selected from the group consisting of: hydroxyproline, proline, ornithine and alpha-aminobutyric acid, that can be used for prevention or treatment of a subject at risk to develop insulin resistance and diabetes starting in childhood or young adulthood. In addition, the nutritional composition of the invention can be used in the formulation of a consumable diet product, food or beverage product, supplement or food fortification, for the metabolic health in children by promoting healthy musculoskeletal growth and development in childhood.

### BACKGROUND OF THE INVENTION

Management of pre-diabetes and Type-2 Diabetes (T2D) in childhood and adolescence has become critical as it affects 1.8 billion adolescents worldwide, which is approximately 25% world population, and about 42 million children under the age of 5, who are overweight or obese. Nutrition has a pivotal role to play since both pre-diabetes and T2D are largely preventable and closely linked to lifestyle, dietary intake and exercise.

Pre-diabetes and diabetes in children and adolescents differ from adults in many physiological and metabolic aspects, for example, due to differences in sexual maturity and growth, neurologic vulnerability to hypoglycaemia, and ability to provide self-care. Insulin resistance (IR) is subject to marked variations, being particularly influenced by pubertal timing as well as both changing body composition and physical activity. Childhood and pubertal IR may result from increased metabolic and physiological requirements, including the effects of increased growth hormone secretion, either direct and/or via the action of IGF-1 (Pinkney et al., 2014).

However, there is less available data in children and adolescents compared to adults, thus the understanding of the underlying mechanisms that link obesity and IR is incomplete.

In the context of metabolic health, childhood and adolescence, obesity introduces a significant disturbance into normal growth and pubertal patterns (Sandhu et al., 2006; Marcovecchio and Chiarelli, 2013). Recent analysis from the Earlybird study has demonstrated the important influences on IR related to age and gender in puberty (Jeffery et al., 2018), which differs in many ways with the adult phenotype (Jeffery et al., 2012). The study exemplified how IR starts to rise in mid-childhood, some years before puberty, with more than 60% of the variation in IR prior to puberty remaining unexplained. In addition, conventional markers to detect diabetes, and to identify individuals at high risk of developing diabetes, and for adult metabolic disease risk, such as HbA1c, lose sensitivity and specificity for pediatric applications, suggesting that other factors influence the variance of these markers in adolescents (Hosking et al., 2014).

One potentially important factor currently being studied is the role of excess body weight during childhood. This can also influence pubertal development through effects on timing of pubertal onset and hormone levels (Marcovecchio and Chiarelli, 2013). The interactions of adiposity with puberty is complex and gender-specific. Moreover, in girls, higher level of IR limit further gain in body fat in the long term, an observation potentially consistent with the concept of IR as a mechanism of insulin desensitization as an adaptive response to weight gain (Hosking et al., 2011). Recently, weight gain and impaired glucose metabolism were shown to be predicted by inefficient subcutaneous fat cell lipolysis (Amer et al., 2018). Adipocyte mobilization of fatty acids (lipolysis) is instrumental for energy expenditure. Lipolysis displays both spontaneous (basal) and hormone-stimulated activity. Thus, inefficient lipolysis (high basal/low stimulated) is linked to future weight gain and impaired glucose metabolism and may constitute a treatment target.

The role of resting energy expenditure and weight gain in children is subject to controversy, with interest in studying the influence of puberty on long term body composition. Obesity develops when energy intake is greater than energy expenditure, the excess energy being stored mainly as fat in adipose tissue. Body weight loss and prevention of weight gain can be achieved by reducing energy intake or bioavailability, increasing energy expenditure, and/or reducing storage as fat. However, overweight subjects or subjects at risk of becoming overweight often need nutritional assistance for better managing their body weight, e.g. through increasing satiety and/or reducing body weight gain.

To address these evidence gaps, the EarlyBird study was designed as a longitudinal cohort study of healthy children with the express intent to investigate the influences of anthropometric, clinical and metabolic processes on glucose and insulin metabolism during childhood and adolescence. The EarlyBird cohort is a non-interventional prospective study of 300 healthy UK children followed-up annually throughout childhood. The investigators tackled the challenging task of integrating and correlating the temporal variations of these different data types in the Earlybird childhood cohort from age 5 to age 20, including anthropometric, clinical and serum biochemical (metabonomic) data.

The present inventors observed that only few and specific amino acid metabolites were associated with IR development throughout childhood and early adulthood in this cohort of healthy children. The population of children overweight or obese at age 5, further developed excessive fat mass gain and body weight gain throughout puberty and adolescence, and have higher HOMA-IR than in other children. In the Earlybird cohort, overweight children at age 5 remain overweight throughout childhood, and will acquire a high IR status from age 10 during pubertal development and development of additional fat mass. The present inventors identified association with hydroxyproline, proline, ornithine and alpha-aminobutyric acid status, which may be indicative of potential deregulation of oxidative stress, collagen metabolism and muscle functions during growth and development, concomitant or contributing to IR development.

Further, in this regard, the combination of these compounds may have additional effects to promote healthy fat and lean mass metabolism during growth and development.

### SUMMARY OF THE INVENTION

### Nutritional Composition

In some embodiments, there is provided a composition, particularly a nutritional composition, for use in a child or adolescent.

In some embodiments, there is provided a composition, particularly a nutritional composition, for use in a child or adolescent in management of glucose metabolism.

In some embodiments, there is provided a composition, particularly a nutritional composition, for use in a child or adolescent with insulin resistance (IR) or risk of developing insulin resistance (IR).

In some embodiments, there is provided a composition, particularly a nutritional composition, for use in a child or adolescent with pre-diabetes or risk of developing pre-diabetes.

In some embodiments, there is provided a composition, particularly a nutritional composition, for use in a child or adolescent to promote healthy fat mass and healthy lean skeletal muscle mass for their age and sex.

In some embodiments, there is provided a composition, particularly a nutritional composition, for use in a method of treatment of obesity in a child or adolescent.

In some embodiments, there is provided a composition, particularly a nutritional composition, for use in a method of prevention of obesity in a child or adolescent.

In some embodiments, there is provided a composition, particularly a nutritional composition for use in a child or adolescent as a part of a dietary intervention for weight loss or weight maintenance. In one embodiment, the nutritional composition is part of a dietary intervention where caloric intake is restricted.

In several embodiments, the nutritional composition comprises one or more amino acids selected from the group consisting of: hydroxyproline, proline, ornithine and alpha-aminobutyric acid; or mixtures thereof for use in a child or adolescent.

In one embodiment, the nutritional composition contains at least hydroxyproline for use in a child or adolescent.

In one embodiment, the nutritional composition contains at least hydroxyproline and proline for use in a child or adolescent.

In one embodiment, the nutritional composition contains at least hydroxyproline, proline and ornithine for use in a child or adolescent.

In one embodiment, the nutritional composition contains at least hydroxyproline, proline, ornithine and alpha-aminobutyric acid for use in a child or adolescent.

In a preferred embodiment, the nutritional composition of the invention improves insulin resistance.

In a preferred embodiment, the nutritional composition of the invention improves glucose tolerance or fasting glucose levels.

In a preferred embodiment, the nutritional composition of the invention helps growth and maintenance of muscle mass and muscle strength in a child or adolescent subject.

In a preferred embodiment, the nutritional composition of the invention helps to limit excess fat mass gain during growth and development of a child or adolescent subject.

In one embodiment, the nutritional composition containing one or more amino acids of the invention is formulated together with (i) a source of fats, (ii) a source of carbohydrates, and (iii) a source of proteins into a diet product and is provided during the low caloric dietary intervention to help weight loss.

In one embodiment, the nutritional composition containing the one or more amino acids are administered simultaneously, sequentially or separately to a subject.

In another embodiment, the nutritional composition containing the one or more amino acids are administered together formulated in a diet product containing (i) a source of fats, (ii) a source of carbohydrates, and (iii) a source of proteins.

In one embodiment, the nutritional formulation of the diet product containing the nutritional composition of the invention with the one or more amino acids of the invention, provides not less than 40% of the subject's average daily calorie intake during the low caloric dietary intervention.

In one embodiment, the nutritional formulation of the diet product containing the nutritional composition of the invention with the one or more amino acids of the invention, provides not less than 70% of the subject's average daily calorie intake during the low caloric dietary intervention.

In one embodiment, the nutritional formulation of the diet product containing the nutritional composition of the invention with the one or more amino acids of the invention, provides not less than 80% of the subject's average daily calorie intake during the low caloric dietary intervention.

In one embodiment, the nutritional formulation of the diet product containing the nutritional composition of the invention with the one or more amino acids of the invention, provides not less than 90% of the subject's average daily calorie intake during the low caloric dietary intervention.

In one embodiment, the nutritional formulation of the diet product containing the nutritional composition of the invention with the one or more amino acids of the invention, provides 100% of the subject's daily calorie intake during the low caloric dietary intervention.

In one embodiment, the subject's average daily calorie intake is about 600 kcal to about 1500 kcal during the low dietary intervention.

In another embodiment, the nutritional composition containing the one or more amino acids of the invention is formulated together with (i) a source of fats, (ii) a source of carbohydrates, and (iii) a source of proteins into a product and is provided after weight loss to help maintain the weight lost.

In one embodiment, the nutritional formulation of the diet product containing the nutritional composition of the invention with the one or more amino acids of the invention, provides 40% of the subject's daily calorie intake after the low caloric dietary intervention during the weight maintenance period.

In one embodiment, the nutritional formulation of the diet product containing the nutritional composition of the invention with the one or more amino acids of the invention, provides 30% of the subject's daily calorie intake after the low caloric dietary intervention during the weight maintenance period.

In one embodiment, the nutritional formulation of the diet product containing the nutritional composition of the invention with the one or more amino acids of the invention, provides 20% of the subject's daily calorie intake after the low caloric dietary intervention during the weight maintenance period.

In one embodiment, the nutritional formulation of the diet product containing the nutritional composition of the invention with the one or more amino acids of the invention, provides 10% of the subject's daily calorie intake after the low caloric dietary intervention during the weight maintenance period.

In another embodiment, after the ideal weight has been achieved, the subject may take the nutritional formulation of the diet product containing the nutritional composition of the invention with the one or more amino acids of the invention, for up to about 26 weeks during the weight maintenance period.

In one embodiment, the nutritional composition provides 1 g to 10 g of the subject's average daily hydroxyproline intake during the dietary intervention or after dietary intervention during the weight maintenance period.

In one embodiment, the nutritional composition provides 1 g to 10 g of the subject's average daily proline intake during the dietary intervention or after dietary intervention during the weight maintenance period.

In one embodiment, the nutritional composition provides 1 g to 10 g of the subject's average daily ornithine intake during the dietary intervention or after dietary intervention during the weight maintenance period.

In one embodiment, the nutritional composition provides 1 g to 10 g of the subject's average daily alpha-aminobutyric acid intake during the dietary intervention or after dietary intervention during the weight maintenance period.

In one embodiment, the subject is diagnosed as “obese” by measuring BMI and comparing to other children or adolescents of the same age and the levels of the amino acids at fasting: hydroxyproline, proline, ornithine levels in the blood, particularly in the red blood cells and when any of these are higher than the reference values, and alpha-aminobutyric acid lower than the reference values, the subject may be considered as being a candidate for the nutritional composition of the invention.

In one embodiment, the subject is diagnosed as being “insulin resistant” or at risk of being “insulin resistant” using one of the known measurements such as: fasting insulin levels, glucose tolerance test and Matsuda index, Homeostatic Model Assessment (HOMA), Quantitative insulin sensitivity check index (QUICKI), hyperinsulinemic euglycemic clamp or a modified insulin suppression test; and measuring the levels of the amino acids at fasting: hydroxyproline, proline, ornithine levels in the blood, particularly in the red blood cells and when any of these are higher than the reference values, and alpha-aminobutyric acid lower than the reference values, the subject may be considered as being a candidate for the nutritional composition of the invention.

In one embodiment, the subject is diagnosed as being “pre-diabetic” or at risk of being “pre-diabetic” using one of the known measurements, such as measurement of Impaired glucose tolerance (IGT) and/or Impaired fasting glucose (IFG); and measuring the levels of the amino acids at fasting: hydroxyproline, proline, ornithine levels in the blood, particularly in the red blood cells and when any of these are higher than the reference values, and alpha-aminobutyric acid lower than the reference values, the subject may be considered as being a candidate for the nutritional composition of the invention.

### Amino Acids

In several embodiments, the nutritional composition of the inventions comprises one or more amino acids selected from the group consisting of: hydroxyproline, proline, ornithine and alpha-aminobutyric acid.

### Methods and Uses

In several embodiments, the nutritional composition of the invention is suitable for use in glucose management.

In several embodiments, the nutritional composition of the invention is suitable for use by children and adolescents.

In one embodiment, the nutritional composition of the invention is suitable for use in a method of preventing or treating insulin resistance in a child or adolescent.

In one embodiment, the nutritional composition of the invention is suitable for use in a method of preventing or treating pre-diabetes in a child or adolescent.

In one embodiment, the nutritional composition of the invention is suitable for use in a child or adolescent to promote healthy fat mass and healthy lean skeletal muscle mass for their age and sex.

In one embodiment, the nutritional composition of the invention is suitable for use in a child or adolescent as a part of a dietary intervention for weight loss or weight maintenance.

In one embodiment, the dietary intervention is the administration of a nutritional composition of the invention as a part of a restricted or low calorie diet.

In one embodiment, the low calorie diet comprises a decreased consumption of fat.

In one embodiment, the low calorie diet comprises an increase in consumption of low fat foods. In one embodiment, the nutritional composition of the invention and the low calorie diet provide the subject with on average between 600 to about 1500 kcal per day.

In one embodiment, the low calorie diet is a reduction in calorie intake by on average not less less than 10% per day compared to average calorie intake per day before the administration of the low calorie diet.

In one embodiment, the low calorie diet is a reduction in calorie intake by on average not less less than 15% per day compared to average calorie intake per day before the administration of the low calorie diet.

In one embodiment, the low calorie diet is a reduction in calorie intake by on average not less less than 20% per day compared to average calorie intake per day before the administration of the low calorie diet.

In one embodiment, the low calorie diet is a reduced in calorie intake compared to average calorie intake per day in the 2 week period immediately before the administration of the low calorie diet.

In one embodiment, the low calorie diet is administered for a duration of up to 12 weeks. In another embodiment, the low caloric diet is administered for a duration from 6 to 12 weeks.

In one embodiment, the method of weight loss using a nutritional composition of the invention is suitable for administration of a low calorie diet

The method of the invention may further comprise determining one or more anthropometric measures and/or lifestyle characteristics of the subject to determine whether the subject is in need of the composition of the invention to achieve weight loss or to maintain weight loss.

The anthropometric measure may be selected from the group consisting of gender, weight, height, age.

The lifestyle characteristic may be, for example, whether the subject performs on average less than the recommended daily levels of exercise.

In one embodiment, the diet product comprises Optifast® or Modifast® with the addition of the one or more amino acids of the invention.

### Kit of Parts

There is provided a kit of parts for use in accordance with the invention, said kit comprising the parts: (i) a diet product; (ii) the nutritional composition containing one or more amino acids of the invention and (iii) instructions for their use.

In one embodiment, the diet product comprises one or more of (i) a source of fats; (ii) a source of carbohydrates; and (iii) a source of proteins.

In one embodiment, the diet product comprises two or more of (i) a source of fats, (ii) a source of carbohydrates, and (iii) a source of proteins.

In one embodiment, the kit of parts containing (i) the diet product and (ii) the nutritional composition containing the one or more amino acids with (iii) instructions for their use are administered simultaneously, sequentially or separately to a diet resistant obese or weight loss resistant obese subject.

In one embodiment, the nutritional composition containing the one or more amino acids are administered with the specified one or more amino acids administered simultaneously, sequentially or separately to a subject.

In another embodiment, the nutritional composition containing the one or more amino acids are administered together formulated together in a diet product containing (i) a source of fats, (ii) a source of carbohydrates, and (iii) a source of proteins.

In one embodiment, the kit is for use in the improvement of weight loss during a dietary intervention, particularly a low caloric dietary intervention.

In one embodiment, the kit is for use in maintenance of weight loss after a dietary intervention, particularly a low caloric dietary intervention.

There is also provided a method of making a kit of parts of the invention.

### DETAILED DESCRIPTION OF THE INVENTION

All percentages are by weight of the total weight of the composition unless expressed otherwise. Similarly, all ratios are by weight unless expressed otherwise. When reference is made to the pH, values correspond to pH measured at 25° C. with standard equipment. As used herein, “about,” “approximately” and “substantially” are understood to refer to numbers in a range of numerals, for example the range of −10% to +10% of the referenced number, preferably −5% to +5% of the referenced number, more preferably −1% to +1% of the referenced number, most preferably −0.1% to +0.1% of the referenced number.

Furthermore, all numerical ranges herein should be understood to include all integers, whole or fractions, within the range. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth.

As used herein and in the appended claims, the singular form of a word includes the plural, unless the context clearly dictates otherwise. Thus, the references “a,” “an” and “the” are generally inclusive of the plurals of the respective terms. For example, reference to “an ingredient” or “a method” includes a plurality of such “ingredients” or “methods.” The term “and/or” used in the context of “X and/or Y” should be interpreted as “X,” or “Y,” or “X and Y.” Similarly, “at least one of X or Y” should be interpreted as “X,” or “Y,” or “both X and Y.”

Similarly, the words “comprise,” “comprises,” and “comprising” are to be interpreted inclusively rather than exclusively. Likewise, the terms “include,” “including” and “or” should all be construed to be inclusive, unless such a construction is clearly prohibited from the context. However, the embodiments provided by the present disclosure may lack any element that is not specifically disclosed herein. Thus, a disclosure of an embodiment defined using the term “comprising” is also a disclosure of embodiments “consisting essentially of” and “consisting of” the disclosed components. “Consisting essentially of” means that the embodiment comprises more than 50 wt. % of the identified components, preferably at least 75 wt. % of the identified components, more preferably at least 85 wt. % of the identified components, most preferably at least 95 wt. % of the identified components, for example at least 99 wt. % of the identified components.

Where used herein, the term “example,” particularly when followed by a listing of terms, is merely exemplary and illustrative, and should not be deemed to be exclusive or comprehensive. Any embodiment disclosed herein can be combined with any other embodiment disclosed herein unless explicitly indicated otherwise.

The relative terms “improved,” “increased,” “enhanced” and the like refer to the effects of the composition comprising both one or more amino acids relative to a composition without the one or more amino acids of the invention or with less of the one or more amino acids, but otherwise identical.

The terms “diet product” and “nutritional composition” and “nutritional formulation” mean a product or composition comprising at least one amino acid of the invention that is intended for ingestion by an individual such as a human and provides at least one nutrient to the individual.

The compositions of the present disclosure, including the many embodiments described herein, can comprise, consist of, or consist essentially of the essential elements and limitations described herein, as well as any additional or optional ingredients, components, or limitations described herein or otherwise useful in a diet.

As used herein, “complete nutrition” contains sufficient types and levels of macronutrients (protein, fats and carbohydrates) and micronutrients to be sufficient to be a sole source of nutrition for the animal to which the composition is administered. Individuals can receive 100% of their nutritional requirements from such complete nutritional compositions.

“Animal” includes, but is not limited to, mammals, which includes but is not limited to rodents, aquatic mammals, domestic animals such as dogs and cats, farm animals such as sheep, pigs, cows and horses, and humans. Where “animal,” “mammal” or a plural thereof is used, these terms also apply to any animal that is capable of the effect exhibited or intended to be exhibited by the context of the passage. As used herein, the term “subject” or “patient” is understood to include an animal, for example a mammal, and preferably a human that is receiving or intended to receive treatment, as treatment is herein defined. While the terms “individual” and “patient” are often used herein to refer to a human, the present disclosure is not so limited.

Accordingly, the terms “subject”, “individual” and “patient” refer to any animal, mammal or human that can benefit from the methods and compositions disclosed herein.

The following terms are used throughout the specification to describe the different early life stages of a subject of the invention, particularly a human subject:

The term “Infant” or “Newborn” is considered to be a human subject during the first month after birth; an “Infant” may be also defined as a human subject between 1 and 23 months of age inclusive.

The term “Child” or “Preschooler” is considered to be a human subject between the ages of 2 and 5 inclusive, i.e. from the subject's 2^{nd} birthday up to and including the day before their 6th birthday. A “Child” may also be considered to be a human subject between the ages of 6 and 12 inclusive.

The term “Prepuberty” is considered to be a human subject age 6 or 7.

The term “Mid-childhood” is considered to be a human subject age 7 or 8.

The term “Adolescent” or “Adolescence is considered to be a human subject between the ages of 13 and 18 inclusive.

The term “Adulthood” is considered to be a human subject 19 years old and above.

The term Insulin resistance (IR) is a pathological condition in which cells fail to respond normally to the hormone insulin. The body produces insulin when glucose starts to be released into the bloodstream from the digestion of carbohydrates (primarily) in the diet. Under normal conditions of insulin reactivity, this insulin response triggers glucose being taken into body cells, to be used for energy, and inhibits the body from using fat for energy, thereby causing the concentration of glucose in the blood to decrease as a result, staying within the normal range even when a large amount of carbohydrates is consumed. During insulin resistance, however, excess glucose is not sufficiently absorbed by cells even in the presence of insulin, thereby causing an increase in the level of blood sugar. IR is one of the factors involved in type 2 Diabetes and Pre-diabetes.

IR can be diagnosed through different means:
Fasting insulin levels: A fasting serum insulin level greater than 25 mIU/L or 174 pmol/L is considered insulin resistance
Glucose tolerance test and Matsuda index
Homeostatic Model Assessment (HOMA), the normal reference range for HOMA-IR differs depending on ethnicity and gender, and must be defined for each population.
Quantitative insulin sensitivity check index (QUICKI)
Hyperinsulinemic euglycemic clamp
Modified insulin suppression test

The term “pre-diabetes” describes a condition in which fasting blood glucose levels are equal or higher than 5.6 mmol/L of blood plasma, although not high enough to be diagnosed with type 2 diabetes. Pre-diabetes has no signs or symptoms. People with pre-diabetes have a higher risk of developing type 2 diabetes and cardiovascular (heart and circulation) disease. Without sustained lifestyle changes, including healthy eating, increased activity and losing weight, approximately one in three people with pre-diabetes will go on to develop type 2 diabetes.

There are two pre-diabetic conditions:

Impaired glucose tolerance (IGT) is where blood glucose levels are equal or higher than 5.6 mmol/L of blood plasma but not high enough to be classified as diabetes. Impaired glucose tolerance is defined as two-hour glucose levels of 140 to 199 mg per dL (7.8 to 11.0 mmol) on the 75-g oral glucose tolerance test, so levels for diabetes is above 11 mmol in ogtt.

Impaired fasting glucose (IFG) is where blood glucose levels are escalated in the fasting state but not high enough to be classified as diabetes. Impaired fasting glucose is defined as glucose levels of 100 to 125 mg per dL (5.6 to 6.9 mmol per L) in fasting patients. So diabetes is above 6.9 mmol.

It is possible to have both Impaired Fasting Glucose (IFG) and Impaired Glucose Tolerance (IGT).

As used herein, the term “reference value” can be defined as the average value measured in biofluid samples of a substantially healthy normal glycaemic population. Said population may have an average fasting glucose level of less than 5.6 mmol/L. The average age of said population is preferably substantially the same as that of the subject. The average BMI sds of said population is preferably substantially the same as that of the subject. The average physical activity level of said population is preferably substantially the same as that of the subject. Said population may be of substantially the same race as the human subject. Said population may number at least 2, 5, 10, 100, 200, 500, or 1000 individuals. Said population may be substantially the same breed when the subject is a pet.

The term “high levels of glucose” or “high glucose levels” is defined as equal to or higher than 5.6 mmol/L as measured in a biofluid sample of a subject.

The term “biofluid” can be, for example, human blood (particularly human blood serum, human blood plasma), urine or interstitial fluids.

“Overweight” is defined for an adult human as having a BMI between 25 and 30. “Body mass index” or “BMI” means the ratio of weight in kg divided by the height in metres, squared. “Obesity” is a condition in which the natural energy reserve, stored in the fatty tissue of animals, in particular humans and other mammals, is increased to a point where it is associated with certain health conditions or increased mortality. “Obese” is defined for an adult human as having a BMI greater than 30. “Normal weight” for an adult human is defined as a BMI of 18.5 to 25, whereas “underweight” may be defined as a BMI of less than 18.5. Body mass index (BMI) is a measure used to determine childhood overweight and obesity in children and teens.

Overweight in children and teens is defined as a BMI at or above the 85th percentile and below the 95th percentile for children and teens of the same age and sex. Obesity is defined as a BMI at or above the 95th percentile for children and teens of the same age and sex. Normal weight in children and teens is defined as a BMI at or above the 5th percentile and below the 85th percentile for children and teens of the same age and sex. Underweight in children and teens is defined as below the 5th percentile for children and teens of the same age and sex. BMI is calculated by dividing a person's weight in kilograms by the square of height in meters. For children and teens, BMI is age- and sex-specific and is often referred to as BMI-for-age. A child's weight status is determined using an age- and sex-specific percentile for BMI rather than the BMI categories used for adults. This is because children's body composition varies as they age and varies between boys and girls. Therefore, BMI levels among children and teens need to be expressed relative to other children of the same age and sex.

The term “subject” is preferably a human subject or can be a pet subject e.g. a cat a dog. In one embodiment, the subject is a male subject. In one embodiment, the subject is a female subject.

The term “substantially” is taken to mean 50% or greater, more preferably 75% or greater, or more preferably 90% or greater. The term “about” or “approximately” when referring to a value or to an amount or percentage is meant to encompass variations of in some embodiments ±20%, in some embodiments ±10%, in some embodiments ±5%, in some embodiments ±1%, in some embodiments ±0.5%, and in some embodiments ±0.1% from the specified value, amount or percentage.

### Composition and Uses Thereof

The “composition”, particularly the “nutritional composition” and/or “nutritional formulation” into a “diet product” containing amino acids of the invention is suitable for use in a number of conditions where glucose management is necessary.

In several embodiments, said composition of the invention is useful in a method of glucose management in a child or adolescent subject in need comprising the steps of:

i) providing the subject a nutritional composition according to the invention; and
ii) administering the nutritional composition to said subject.

In several embodiments, said composition of the invention is useful in a method of glucose management in a child or adolescent subject wherein said subject is determined as being insulin resistant or at risk of being insulin resistant.

In several embodiments, said composition of the invention is useful in a child or adolescent subject wherein insulin resistant or risk of being insulin resistant is determined according to measurement of the subject for: fasting insulin levels, glucose tolerance test and Matsuda index, Homeostatic Model Assessment (HOMA), Quantitative insulin sensitivity check index (QUICKI), hyperinsulinemic euglycemic clamp or a modified insulin suppression test compared to a reference age and sex cohort.

In several embodiments, said composition of the invention is useful in a method of glucose management in a child or adolescent subject in need wherein said subject is determined as being pre-diabetic or at risk of being pre-diabetic.

In several embodiments, said composition of the invention is useful in a method of glucose management in a child or adolescent subject wherein said condition of pre-diabetic or risk of being pre-diabetic is determined according to measurement of the subject using a Impaired glucose tolerance (IGT) test and/or Impaired fasting glucose (IFG) test compared to a reference age and sex cohort.

In several embodiments, said composition of the invention is useful in a method of glucose management in an obese child or adolescent subject during weight loss during a dietary intervention and maintenance of weight after weight loss. Weight loss can be measured by any technique known to the person skilled in the art.

“Weight loss” as defined herein may refer to a reduction in parameters such as weight (e.g. in kilograms), body mass index (kgm-2), waist-hip ratio (e.g. in centimetres), fat mass (e.g. in kilograms), hip circumference (e.g. in centimetres) or waist circumference (e.g. in centimetres).

Weight loss may be calculated by subtracting the value of one or more of the aforementioned parameters at the end of an intervention from the value of said parameter at the onset of the intervention (e.g. a use according to the present invention).

The degree of weight loss may be expressed as a percentage change of one of the aforementioned weight phenotype parameters (e.g. a percentage change in a subject's body weight (e.g. in kilograms) or body mass index (kgm-2). For example, a subject may lose at least 10% of their initial body weight, at least 8% of their initial body weight, or at least 5% of their initial body weight. By way of example only, a subject may lose between 5 and 10% of their initial body weight.

In one embodiment, a degree of weight loss of at least 10% of initial body weight results in a considerable decrease in risk for obesity related co-morbidities.

“Maintaining weight loss” as defined herein may refer to the maintenance in parameters such as weight (e.g. in kilograms), body mass index (kgm-2), waist-hip ratio (e.g. in centimetres) fat mass (e.g. in kilograms), hip circumference (e.g. in centimetres) or waist circumference (e.g. in centimetres) or maintenance of fat mass following an intervention such as a dietary intervention.

Typically, maintenance of weight loss occurs after a period of attaining weight loss.

In one aspect, the present invention provides the non-therapeutic use of the nutritional composition of the invention to maintain a healthy body composition after a period weight loss.

The degree of weight maintenance may be calculated by determining the change in one or more of the aforementioned parameters during a period of time. The period of time may be for example at least 12, 15, 20, 26, 30, 36, 40, 46 or 50 weeks.

The degree of weight maintenance may be expressed as the weight regained during a period following attainment of weight loss, for example as a percentage of the weight lost during attainment of weight loss.

In one aspect, the present invention provides a nutritional composition and/or formulation into a diet product of the invention for use in attaining or maintaining weight loss in a subject. The nutritional composition and/or formulation into a diet product of the invention can also improve fat mass loss and BMI, which can be measured by any technique known to the person skilled in the art.

### Subject

The subject of the invention is preferably a mammal. In a preferred embodiment, the subject is a human. Most preferably, the subject is a child or adolescent. In one preferred embodiment, the subject may be a human child between the ages of 6 and 12 inclusive. In another preferred embodiment, the subject may be a human adolescent between the ages of 13 and 18 inclusive.

“Overweight” is defined for an adult human as having a BMI between 25 and 30. “Body mass index” or “BMI” means the ratio of weight in kg divided by the height in metres, squared. “Obesity” is a condition in which the natural energy reserve, stored in the fatty tissue of animals, in particular humans and other mammals, is increased to a point where it is associated with certain health conditions or increased mortality. “Obese” is defined for an adult human as having a BMI greater than 30. “Normal weight” for an adult human is defined as a BMI of 18.5 to 25, whereas “underweight” may be defined as a BMI of less than 18.5.

Obesity related disorder refers to any condition which an obese individual is at an increased risk of developing.

The obesity-related disorder may be diabetes (e.g. type 2 diabetes), stroke, high cholesterol, cardiovascular disease, insulin resistance, coronary heart disease, metabolic syndrome, hypertension or fatty liver.

The nutritional composition of the invention is suitable for use in a child or adolescent to promote healthy fat mass and healthy lean skeletal muscle mass for their age and sex.

“Fat mass” refers to the portion of a subject's body which is composed of fat. Fat mass may be determined using a wide range of methods, for example caliper-based measurements of skinfold thickness, Dual energy X-ray absorptiometry, CT or MRI scanning or bioelectrical impedance analysis.

Reducing fat mass may mean that fat mass is reduced by at least 1%, at least 2%, at least 5%, at least 10%, at least 15%, at least 20%, at least 30%, at least 40% or at least 50%.

“Lean mass” or “lean body mass” refers to the part of body composition that is defined as the difference between total body weight and body fat weight. This means that it counts the mass of all organs except body fat, including bones, muscles, blood, skin, and everything else. Maintaining lean body mass is important for optimal metabolism, normal physical activity and good health.

Substantially maintaining lean mass may mean that lean mass alters by, for example, less than 7%, less than 5%, less than 4%, less than 3%, less than 2% or less than 1% following or during an intervention.

Preferably, the majority of weight loss is due to a reduction in non-lean mass or fat mass rather than lean mass.

Preferably the degree of weight loss is represented by the number of BMI units lost, where BMI loss=((BMI1−BMI2)*100)/BMI1, wherein BMI1 is the body mass index of the subject before the dietary intervention and BMI2 is the predicted body mass index of the subject after the dietary intervention.

### Dietary Intervention

The term “dietary intervention” is taken to mean an external factor applied to a subject which causes a change in the subject's diet. In a preferred embodiment, the “dietary intervention” includes the nutritional composition of the invention. In another preferred embodiment, the dietary intervention is a restricted calorie diet including a nutritional composition of the present invention.

The estimated needs for young children range from 1,000 to 2,000 calories per day, and the range for older children and adolescents varies from about 1,400 to 3,200 calories per day, with boys generally having higher calorie needs than girls, especially after the onset of puberty.

A “restricted calorie diet” comprises an average calorie intake of about 600 to about 1500 kcal/day, more preferably an average of about 600 to about 1200 kcal/day, more preferably an average of about 700 to about 900 kcal/day, most preferably an average of about 800 kcal/day.

In one embodiment, the low calorie diet may comprise a predetermined amount of vegetables per day, preferably up to about 400 g vegetables/day, e.g. about 200 g vegetables/day. Low fat foods may include wholemeal flour and bread, porridge oats, high-fibre breakfast cereals, wholegrain rice and pasta, vegetables and fruit, dried beans and lentils, baked potatoes, dried fruit, walnuts, white fish, herring, mackerel, sardines, kippers, pilchards, salmon and lean white meat.

The restricted calorie diet may comprise administration of at least one “diet product”. The “diet product” may be formulated to be a meal replacement product or a supplement product which may e.g. suppress the subject's appetite. The diet product can include food products, beverage products, pet food products, food supplements, nutraceuticals, food additives or nutritional formulas.

In one embodiment, the diet product may comprise a product, for example, Optifast® or Modifast® with formulation with the amino acids of the invention.

The “diet product” may be supplemented with three portions of non-starchy vegetables such that the total energy intake is about 2.5 MJ (600 kcal/day). This may be further supplemented with at least 2 L of water or other energy free beverages per day.

In another embodiment, the “diet product” may comprise, for example, a nutritional formulation which comprises a nutritional composition of the amino acids of the invention and up to at least 46.4% carbohydrate, 32.5% protein and 20.1% with fat, vitamins, minerals and trace elements; which may be supplemented with three portions of non-starchy vegetables such that the total energy intake is about 2.5 MJ (600 kcal/day) per serving and up to a total of 1500 kcal/day. This may be further supplemented with at least 2 L of water or other energy free beverages per day.

In one embodiment, the restricted calorie diet has a duration of up to 12 weeks. Preferably the low calorie diet has a duration of between 6 and 12 weeks, preferably between 8 and 10 weeks, e.g. 8 weeks.

### Amino Acids

The term “amino acid” is a generic term for an organic compound having both an amino group (—NH2) and a carboxyl group (—COOH).

In several embodiments of the invention, specific amino acids and their derivatives are described.

### Proline

In several embodiments of the invention, “proline” or a “proline derivative” is defined in the nutritional composition, methods and uses. Proline is a non-essential amino acid that is synthesized from glutamic acid. It is an essential component of collagen and is important for proper functioning of joints and tendons.

“Proline” is an amino acid which is also known as L-proline; L-(−)-Proline; (S)-Pyrrolidine-2-carboxylic acid; (2S)-pyrrolidine-2-carboxylic acid; (−)-Proline, (−)-(S)-Proline; Prolinum; H-Pro-OH,2-pyrrolidinecarboxylic acid; (S)-2-Pyrrolidinecarboxylic acid; prolina, (−)-2-Pyrrolidinecarboxylic acid; L-Pyrrolidine-2-carboxylic acid; L-alpha-Pyrrolidinecarboxylic acid; L-Prolin; Carboxypyrrolidine.

A “proline derivative” is preferably an L-proline and/or defined as being selected from the group consisting of: cis-4-hydroxy-L-proline (CHP), trans-4-hydroxy-L-proline (THP), 4-hydroxy-1-methyl-proline, 1-methyl-4-phenylamine carbonyloxy-proline, 1-methyl-4-phenylamine carbonyloxy-proline, cis-4-hydroxymethyl-1-proline, trans-4-hydroxymethyl-D-proline, trans-4-hydroxymethyl-1-proline, trans-4-methyl-1-proline, cis-3-Amino-1-proline, 1-Methyl-4-phenylaminocarbonyl-oxy-proline-ethylester, 1-Methyl-4-phenylaminocarbonyl-oxy-proline-isobutylester, 4-Hydroxy-1-methyl-proline-ethylester, 4-Hydroxy-1-methyl-proline-isobutylester, 4-Hydroxy-1-methyl-proline-ethylester, 4-Hydroxy-proline ethylester, 4-Hydroxy-proline-isobutylester, cis-4-Hydroxy-L-proline-ethylester, Cis-4-Hydroxy-L-proline-iso-butylester, 4-Hydroxy-1,1-dimethyl-proline-ethylester-iodide, Hydroxyproline-ethylester, 4-Hydroxy-1,1-dimethyl-proline-iso-butylester-iodide,4-Hydroxy-1-cyclohexyl-proline-isobutyl-ester, 4-Hydroxy-1-diphenylmethyl-proline-isobutylester-hydrobromide, 4-Hydroxy-1-methyl-proline,4-Hydroxy-1-alkyl-prolineester amide (alkyl: methy, ethyl, propyl, pentyl, hexyl, heptyl, octyl, and nonyl), 4-Hydroxy-1-diphenylmethyl-proline-isobutyl-ester-hydrobromide. This embodiment encompasses the salts, esters, isomers, racemates, enantiomers or pro-drugs thereof.

In one embodiment of the invention, “L-proline” is preferred.

### Hydroxyproline

In several embodiments of the invention, “hydroxyproline” is defined in the nutritional composition, methods and uses. 4-Hydroxyproline is a major component of the protein collagen. Hydroxyproline is produced by hydroxylation of the amino acid proline and is a post-translationally modified, non-essential amino acid. Hydroxyproline and proline play key roles in collagen stability.

“Hydroxyproline” is also known as (2S,4R)-4-Hydroxypyrrolidine-2-carboxylic acid; -Hydroxyproline; or L-hydroxyproline. Hydroxyproline differs from proline by the presence of a hydroxyl (OH) group attached to the gamma carbon atom.

In one embodiment of the invention, “trans-4-hydroxyproline” is preferred.

### Ornithine

In several embodiments of the invention, “Ornithine” is defined in the nutritional composition, methods and uses.

“Ornithine” is also known as 2,5-Diaminopentanoic acid, 2,5-Diaminovalerate, 5-Amino-L-norvaline. L-ornithine is a member of the class of compounds known as L-alpha-amino acids. L-alpha-amino acids are alpha amino acids which have the L-configuration of the alpha-carbon atom.

In one embodiment of the invention, “L-ornithine” is preferred.

### Alpha-Aminobutyric Acid

In several embodiments of the invention, “alpha-aminobutyric acid” is defined in the nutritional composition, methods and uses.

“Alpha-aminobutyric acid” is also known as also known as (S)-2-aminobutanoic acid, homoalanine, 2-AABA, ethylglycine, or L-butyrine, is a member of the class of compounds known as L-alpha-amino acids. L-alpha-amino acids are alpha amino acids which have the L-configuration of the alpha-carbon atom.

In one embodiment of the invention, “alpha-aminobutyric acid” is preferred.

### Diet Product

The composition of the invention can be formulated into a “diet product” to be selected from the group consisting of: a food product, a beverage product, a food supplement, an oral nutritional supplement (ONS), a medical food, and combinations thereof.

The “nutritional composition” or “diet product” is preferably administered to the individual at least two days per week, more preferably at least three days per week, most preferably all seven days of the week; for at least one week, at least one month, at least two months, at least three months, at least six months, or even longer. In some embodiments, the composition is administered to the individual consecutively for a number of days, for example at least until a therapeutic effect is achieved. In an embodiment, the composition can be administered to the individual daily for at least 30, 60 or 90 consecutive days.

The above examples of administration do not require continuous daily administration with no interruptions. Instead, there may be some short breaks in the administration, such as a break of two to four days during the period of administration. The ideal duration of the administration of the composition can be determined by those of skill in the art.

The composition can be any kind of composition that is suitable for human and/or animal consumption. For example, the composition may be selected from the group consisting of food compositions, dietary supplements, nutritional compositions, nutraceuticals, powdered nutritional products to be reconstituted in water or milk before consumption, food additives, medicaments, beverages and drinks. In an embodiment, the composition is an oral nutritional supplement (ONS), a complete nutritional formula, a pharmaceutical, a medical or a food product. In a preferred embodiment, the composition is administered to the individual as a beverage. The composition may be stored in a sachet as a powder and then suspended in a liquid such as water for use.

In some instances where oral or enteral administration is not possible or not advised, the composition may also be administered parenterally.

In some embodiments, the composition is administered to the individual in a single dosage form, i.e. all compounds are present in one product to be given to an individual in combination with a meal. In other embodiments, the composition is co-administered in separate dosage forms, for example at least one component separately from one or more of the other components of the composition.

The diet product can comprise (i) a source of protein, (ii) a source of fat and (iii) a source of carbohydrates in addition to the one or more amino acids of the invention. Further description of the components of the diet product are described below.

### Protein

The term “protein” as used herein includes free form amino acids, molecules between 2 and 20 amino acids (referenced herein as “peptides”), and also includes longer chains of amino acids as well. Small peptides, i.e., chains of 2 to 10 amino acids, are suitable for the composition alone or in combination with other proteins. The “free form” of an amino acid is the monomeric form of the amino acid. Suitable amino acids include both natural and non-natural amino acids. The composition can comprise a mixture of one or more types of protein, for example one or more (i) peptides, (ii) longer chains of amino acids, or (iii) free form amino acids; and the mixture is preferably formulated to achieve a desired amino acid profile/content.

The composition can comprise a protein that provides at least a portion of the one or more amino acids and/or at least a portion of the one or more amino acids, and at least a portion of the protein can be from animal or plant origin, for example dairy protein such as one or more of milk protein, e.g., milk protein concentrate or milk protein isolate; caseinates or casein, e.g., micellar casein concentrate or micellar casein isolate; or whey protein, e.g., whey protein concentrate or whey protein isolate. Additionally or alternatively, at least a portion of the protein can be plant protein such as one or more of soy protein or pea protein.

Mixtures of these proteins are also suitable, for example mixtures in which casein is the majority of the protein but not the entirety, mixtures in which whey protein is the majority of the protein but not the entirety, mixtures in which pea protein is the majority of the protein but not the entirety, and mixtures in which soy protein is the majority of the protein but not the entirety. In an embodiment, at least 10 wt. % of the protein is whey protein, preferably at least 20 wt. %, and more preferably at least 30 wt. %. In an embodiment, at least 10 wt. % of the protein is casein, preferably at least 20 wt. %, and more preferably at least 30 wt. %. In an embodiment, at least 10 wt. % of the protein is plant protein, preferably at least 20 wt. %, more preferably at least 30 wt. %.

Whey protein may be any whey protein, for example selected from the group consisting of whey protein concentrates, whey protein isolates, whey protein micelles, whey protein hydrolysates, acid whey, sweet whey, modified sweet whey (sweet whey from which the caseino-glycomacropeptide has been removed), a fraction of whey protein, and any combination thereof.

Casein may be obtained from any mammal but is preferably obtained from cow milk and preferably as micellar casein.

The protein may be unhydrolyzed, partially hydrolyzed (i.e., peptides of molecular weight 3 kDa to 10 kDa with an average molecular weight less than 5 kDa) or extensively hydrolyzed (i.e., peptides of which 90% have a molecular weight less than 3 kDa), for example in a range of 5% to 95% hydrolyzed. In some embodiments, the peptide profile of hydrolyzed protein can be within a range of distinct molecular weights. For example, the majority of peptides (>50 molar percent or >50 wt. %) can have a molecular weight within 1-5 kDa, or 5-10 kDa, or 10-20 kDa.

### Fats

In an embodiment, the composition includes a source of fat. The source of fat may include any suitable fat or fat mixture. Non-limiting examples of suitable fat sources include vegetable fat, such as olive oil, corn oil, sunflower oil, high-oleic sunflower, rapeseed oil, canola oil, hazelnut oil, soy oil, palm oil, coconut oil, blackcurrant seed oil, borage oil, lecithins, and the like, animal fats such as milk fat; or combinations thereof.

### Carbohydrates

In an embodiment, the composition includes a source of carbohydrates. Any suitable carbohydrate may be used in the composition including, but not limited to, starch (e.g., modified starch, amylose starch, tapioca starch, corn starch), sucrose, lactose, glucose, fructose, corn syrup solids, maltodextrin, xylitol, sorbitol or combinations thereof.

The source of carbohydrates is preferably not greater than 50 energy % of the composition, more preferably not greater than 36 energy % of the composition, and most preferably not greater than 30 energy % of the composition.

### Nutritional Formulation

The composition can be any kind of composition formulated such that it is suitable for human and/or animal consumption.

In a preferred embodiment, the composition is formulated as a nutritional formulation suitable for administration to children or adolescents to ensure compliance of use.

For example, the composition may be selected from the group consisting of food compositions, dietary supplements, nutritional compositions, nutraceuticals, powdered nutritional products to be reconstituted in water or milk before consumption, food additives, medicaments, beverages and drinks. In an embodiment, the composition is an oral nutritional supplement (ONS), a complete nutritional formula, a pharmaceutical, a medical or a food product. In a preferred embodiment, the composition is administered to the individual as a beverage. The composition may be stored in a sachet as a powder and then suspended in a liquid such as water for use.

In some embodiments, the composition is administered to the individual in a single dosage form, i.e. all compounds are present in one product to be given to an individual in combination with a meal. In other embodiments, the composition is co-administered in separate dosage forms, for example at least one component separately from one or more of the other components of the composition.

### EXAMPLES

The following non-limiting examples present scientific data developing and supporting the concept of administering a composition comprising one or more amino acids of the invention, the composition further comprising one or more amino acids in an amount effective for the composition to be useful for a in need of weight loss or for weight maintenance after weight loss. It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

### Example 1: Cohort and Methods Used During the Study

### Study Population

The EarlyBird Diabetes Study incorporates a 1995/1996 birth cohort recruited in 2000/2001 when the children were 5 years old (307 children, 170 boys). The collection of data from the Early Bird cohort is composed of several clinical and anthropometric variables measured on an annual basis from the age of 5 to the age of 16. The study was conducted in accordance with the ethics guidelines of the Declaration of Helsinki II; ethics approval was granted by the Plymouth Local Research Ethics Committee (1999), and parents gave written consent and children verbal assent.

Following a good cohort retention at an age when some young people will begin moving from home to start their own lives. A follow-up study was prepared from June 2013 and began study visits in February 2015 until summer 2016. A total of 178 Earlybird participants completed this follow-up visit as an adult (average age of 20 years old) where data were collected using an adapted study protocol.

### Anthropometric Parameters

BMI was derived from direct measurement of height (Leicester Height Measure; Child Growth Foundation, London, U.K.) and weight (Tanita Solar 1632 electronic scales), performed in blind duplicate and averaged. BMI SD scores were calculated from the British 1990 standards.

Physical activity was measured annually from 5 years to 16 years of age by accelerometry (Acti-Graph [formerly MTI/CSA]). Children were asked to wear the accelerometers for 7 consecutive days at each annual time point, and only recordings that captured at least 4 days were used.

Resting energy expenditure was measured by indirect calorimetry using a ventilated flow through hood technique (Gas Exchange Measurement, Nutren Technology Ltd, Manchester, UK). Performance tests reportedly show a mean error of 0.3±2.0% in the measurement of oxygen consumption and 1.8±1% in that of carbon dioxide production. Measurements were performed in a quiet thermoneutral room (20° C.) after overnight fasting period of at least 6 hours, to minimize any effect attributable to the thermic effect of food. Data were collected for a minimum of 10 minutes and the respiratory quotient (RQ) was calculated as an indicator of basal metabolic rate (BMR).

### Clinical Parameters

Peripheral blood was collected annually into EDTA tubes after an overnight fast and stored at −80° C. Insulin resistance (IR) was determined each year from fasting glucose (Cobas Integra 700 analyzer; Roche Diagnostics) and insulin (DPC IMMULITE) (cross-reactivity with proinsulin, 1%) using the homeostasis model assessment program (HOMA-IR), which has been validated in children.

### Mass-Spectrometry Based Determination of Serum Amino Acids

Blood serum amino acids were quantified on selected samples using an in-house automated quantification method of amino acids in human plasma and serum by UPLC-MS/MS. Tandem mass spectrometry (UPLC-MS/MS) was used for the analysis of amino acids. Separation and analysis were performed on an Accela UHPLC 1250 Pump (Thermo Fisher Scientific Inc., Waltham, Mass., USA) coupled to a TSQ Quantum Vantage triple quadrupole (Thermo Fisher Scientific Inc., Waltham, Mass., USA) equipped with a heated electrospray ionization (H-ESI) source. Chromatographic separation was obtained using gradient elution on a reversed-phase UPLC XSelect HSST3 2.5 μm, 100×2.1 mm I.D. column (Waters Corporation, Milford, Mass., USA). The injection volume was 10 μL and the total run time of analysis was 13 min. Plasma samples were thawed and vortexed for 10 s. A 50-μL volume of plasma was transferred by pipetting into a 1.5-mL microcentrifuge tube. Ten microliters of IS solution was added to plasma. Fifty microliters of TCEP solution and then 140 μL of methanol+1% FA were then added to the mixture. The tubes were placed in a multitube vortexer for 15 min at 1350 rpm at 4° C. and centrifuged at 14,500 rpm for 5 min. The supernatants were pipetted and filtered through a 0.22-μm filter and placed into vials for LC-MS/MS analysis.

### Statistics

Main clinical data are reported in Table 1, and serum amino acid concentrations at the age of 15 and 20 years old are reported in Tables 2 and 3, respectively.

Amongst the clinical variables, the distribution of the outcome variable, IR, was skewed and so log-transformed for analysis.

Data analysis was conducted using cross-sectional analysis using simple linear regression between clinical parameters and and individual metabolites, taking into account age and BMI. Random intercepts were included as well as gender, BMI, and individual metabolites (in separate models) as fixed effects. In addition, multivariate analysis was conducted using SIMCA 14 (Umetrics, Sweden), to apply an Orthogonal Partial Least Squares (OPLS) approach to model simultaneously the associations between all the metabolites and individual clinical parameters.

The present inventors carried out a study on a sub-set of 174 participants at age 15y and 20y. Subjects were chosen on the basis of having a complete both visits, and with clinical and biochemical data available. Blood samples for amino acid analysis were available at age 15 for 165 subjects, and at age 20 for 174 subjects (intersection is 163 subjects). Here, mixed effects modelling was used to assess the association between IR and individual metabolites. Modelling was carried out in R software (www.R-project.org) using the lmer function in the package lme4 (Bates et al., 2015) and p-values calculated using the Satterthwaite approximation implemented in the lmerTest package (Kuznetsova et al., 2016).

TABLE 1
Characteristics of the main cohort at 15 and 20 year of age by gender.

| PARAMETERS | AGE | BOYS | GIRLS |
|---|---|---|---|
| FASTING GLUCOSE | 15 | 5.2 ( 4.5-5.8 ) | 5.1 (4.3-5.6) |
| (MMOL.L⁻¹) | 20 | 4.7 (4-5.5) | 4.5 (3.7-5.6) |
| FASTING INSULIN | 15 | 5.9 (1.5-14.7 ) | 7.8 (1.5-22.1) |
| (MU.L⁻¹) | 20 | 6 (0.1-30.7) | 6.7 (0.5-31.6) |
| HOMA-IR | 15 | 0.9 (0.2-2.2 ) | 1.1 (0.2-3.2) |
| (INSULIN RESISTANCE | 20 | 0.8 (0-2.9 ) | 0.8 (0.1-4) |
| INDEX) | | | |
| HOMA- B | 15 | 77.4 (28.6-151.7 ) | 108.1 (33.3-203.7) |
| (BETA-CELL FUNCTION | 20 | 89.4 (5.2-190.5 ) | 113.1 (14.8-266.6 |
| INDEX) | | | |
| BMI ZSCORE | 15 | 0.4 (−2.9-2.6) | 0.7 (−1.8-3.2) |
| BMI (KG/M⁻²) | 20 | 23.2 (15.4-122.2 ) | 24.4 (13-51.9) |
| MATSUDA INDEX | 20 | 5.8 (1.6-14.5) | 6 (1.2-55.3) |
| Data are median | | | |
| (Min, Max values) | | | |

TABLE 2
Blood serum amino acid concentrations at Year 15

| AMINO ACIDS | | | | |
|---|---|---|---|---|
| (CONCENTRATION IN | | | | |
| MICROMOL.L⁻¹) | MEAN | SD | MIN | MAX |
| L-ARGININE | 93.45 | 18.96 | 48.75 | 155.23 |
| TAURINE | 136.37 | 26.53 | 71.07 | 204.55 |
| L-PHENYLALANINE | 65.17 | 9.44 | 36.57 | 89.59 |
| 1-METHYLHISTIDINE | 3.9 | 0.9 | 2.2 | 6.92 |
| L-ORNITHINE | 81.55 | 18.38 | 39.94 | 121.59 |
| L-ISOLEUCINE | 71.55 | 12.5 | 40.6 | 106.01 |
| L-ASPARAGINE | 54.45 | 8.81 | 30.65 | 76.08 |
| L-GLYCINE | 286.45 | 57.34 | 161.82 | 533.37 |
| L-THREONINE | 147.27 | 30.95 | 85.34 | 232.9 |
| L-ALANINE | 378.87 | 72.04 | 224.1 | 583.63 |
| L-TRYPTOPHAN | 63.61 | 1 1.44 | 30.08 | 106.65 |
| SYMMETRIC | 0.46 | 0.07 | 0.3 | 0.69 |
| DIMETHYLARGININE | | | | |
| (SDMA) | | | | |
| 4-HYDROXYPROLINE | 22 | 11.11 | 7.84 | 60.9 |
| L-HISTIDINE | 90.53 | 12.59 | 56.34 | 129.25 |
| 3-METHYLHISTIDINE | 8.82 | 6.01 | 2 | 27.25 |
| L-LEUCINE | 132.14 | 20.33 | 79.75 | 180.55 |
| L-GLUTAMIC ACID | 51.87 | 14.78 | 23.42 | 95.48 |
| ALPHA-AMINOBLTYRIC | 16.61 | 5.29 | 8.08 | 39.98 |
| ACID (AABA) | | | | |
| L-CITRULLINE | 32.63 | 6.98 | 7.5 | 55.53 |
| ASYMMETRIC | 0.57 | 0.13 | 0.29 | 1.13 |
| DIMETHYLARGININE | | | | |
| (ADMA) | | | | |
| L-PROLINE | 203.64 | 59.32 | 94.43 | 43 1.56 |
| BETA-ALANINE | 4.71 | 1.44 | 2.12 | 10.37 |
| L-METHIONINE | 26.76 | 4.37 | 17.21 | 39.57 |
| L-GLUTAMINE | 722.45 | 89.78 | 478.54 | 974.34 |
| L-TYROSINE | 64.74 | 12.72 | 36.95 | 114.12 |
| VALINE | 230.79 | 35.04 | 149.39 | 318.75 |
| L-SERINE | 153.95 | 26.21 | 81.25 | 236.55 |
| L-ASPARTIC ACID | 20.16 | 5.46 | 8.61 | 37.06 |
| L-LYSINE | 161.4 | 30.47 | 89.33 | 278.38 |

TABLE 3
Blood serum amino acid concentrations at Year 20

| AMINO ACIDS | | | | |
|---|---|---|---|---|
| (CONCENTRATION IN MICROMOL.L⁻¹) | MEAN | SD | MIN | MAX |
| L-ARGININE | 110.87 | 21.67 | 61.36 | 192.26 |
| TAURINE | 105.04 | 25.84 | 52.74 | 195.73 |
| L-PHENYLALANINE | 74.03 | 12.92 | 54.27 | 128.04 |
| 1-METHYLHISTIDINE | 4.47 | 1.32 | 2.08 | 9.76 |
| L-ORNITHINE | 67.32 | 15.74 | 35.58 | 123.86 |
| L-ISOLEUCINE | 76.67 | 18.1 | 44.27 | 140.76 |
| L-ASPARAGINE | 56.36 | 10.92 | 35.2 | 88.51 |
| L-GLYCINE | 277.57 | 64.14 | 176.75 | 562.51 |
| L-THREONINE | 146.82 | 35.97 | 81.65 | 292.05 |
| L-ALANINE | 385.21 | 76.42 | 218.04 | 585.63 |
| L-TRYPTOPHAN | 63.39 | 14.49 | 35.9 | 103.55 |
| SYMMETRIC DIMETHYLARGININE (SDMA) | 0.46 | 0.09 | 0.3 | 0.81 |
| HYDROXYPROLINE | 17.32 | 14.45 | 6.37 | 128.25 |
| L-HISTIDINE | 90.56 | 15.72 | 63.01 | 150.84 |
| 3-METHYLHISTIDINE | 9.66 | 6.88 | 1.97 | 30.02 |
| L-LEUCINE | 143.87 | 30.88 | 80.87 | 238.52 |
| L-GLUTAMIC ACID | 47.65 | 18.21 | 22.22 | 138.97 |
| ALPHA-AMINOBUTYRIC ACID (AABA) | 17.46 | 6.47 | 4.36 | 40.47 |
| L-CITRULLINE | 32.34 | 7.78 | 19.02 | 60.18 |
| ASYMMETRIC DIMETHYLARGININE (ADMA) | 0.52 | 0.11 | 0.26 | 0.83 |
| L-PROLINE | 203.25 | 73.1 1 | 79.57 | 559.65 |
| BETA-ALANINE | 4.26 | 1.52 | 1.96 | 8.89 |
| L-METHIONINE | 27.93 | 5.37 | 18.38 | 42.76 |
| L-GLUTAMINE | 705.7 | 109.5 | 472.97 | 1085.03 |
| L-TYROSINE | 66.54 | 16.21 | 34.11 | 115.19 |
| VALINE | 245.27 | 52.21 | 148.66 | 434.92 |
| L-SERINE | 157.38 | 29.14 | 95.8 | 241.1 |
| L-ASPARTIC ACID | 23.44 | 7.03 | 11.54 | 46.15 |
| L-LYSINE | 164.69 | 37.42 | 97.46 | 296.32 |

### Example 2: Metabolite Concentrations Associated with Glucose Status in Childhood

Relationships between blood amino acids, glycemic and insulin traits were assessed in the subjects at the age of 15 years old using cross-sectional analysis using simple linear regression taking into account gender and BMI (Table 4). Analysis revealed a significant positive association between the nutritional status in Hydroxyproline with fasting insulin and HOMA IR, indicative of insulin sensitivity. Analysis revealed a significant negative association between the nutritional status in alpha-Aminobutyric acid (AABA) with HOMA B parameter, indicative of insulin secretion.

TABLE 4
Association between amino acids and clinical status at Year 15

| LINEAR | | | | |
|---|---|---|---|---|
| REGRESSION, | | | | |
| CORRECTION FOR | METABOLITE | | | |
| GENDER AND BMI | CONCENTRATIONAT | | | ADJUSTED |
| AT AGE 15 | AGE 15 | COEFFICIENT | P-VALUE | P-VALUE |
| FASTING INSULIN | Hydroxyproline | 0.19642 | 0.000211 | 0.00218033 |
| HOMA-IR | Hydroxyproline | 0.02805 | 0.02226 | 0.0471912 |

### Example 3: Metabolite Concentrations Associated with Glucose Status in Adulthood

Relationships between blood amino acids, glycemic and insulin traits were assessed in the subjects at the age of 20 years old using cross-sectional analysis using simple linear regression taking into account gender and BMI (Table 5). Analysis confirmed the reproducibility of the associations seen at the age of 15 between Hydroxyproline with fasting insulin and HOMA IR. In addition, additional associations were observed between insulin and glucose trait with this metabolite, as well as with proline, ornithine and alpha-Aminobutyric acid (AABA).

TABLE 5
Association between amino acids and clinical status at Year 20

| LINEAR | | | | |
|---|---|---|---|---|
| REGRESSION, | | | | |
| CORRECTION FOR | METABOLITE | | | |
| GENDER AND BMI AT | CONCENTRATIONAT | | | ADJUSTED P- |
| AGE 20 | AGE 20 | COEFFICIENT | P-VALUE | VALUE |
| FASTING GLUCOSE | Hydroxyproline | 0.018294 | 0.000165 | 0.000319 |
| FASTING INSULIN | Hydroxyproline | 0.12871 | 0.01958 | 0.0377614 |
| HOMA-IR | Hydroxy proline | 0.031665 | 0.0103 | 0.0188833 |
| MATSUDA INDEX | Hydroxyproline | −0.13609 | 0.0197 | 0.0447727 |
| FASTING GLUCOSE | Proline | 0.001301 | 0.00656 | 0.0121378 |
| FASTING INSULIN | Proline | 0.011504 | 0.0304 | 0.0529548 |
| FASTING GLUCOSE | Ornithine | 0.007591 | 0.00955 | 0.0158257 |
| MATSUDA INDEX | Ornithine | −0.12417 | 0.0121 | 0.0968 |
| FASTING INSULIN | alpha-Aminobuty ric acid | −0.13.342 | 0.021557 | 0.0401406 |
| | (AABA) | | | |
| HOMA-IR | alpha-Aminobutyric acid | −0.035779 | 0.005723 | 0.0112416 |
| | (AABA) | | | |

Additional comparison on the status in these nutrients was conducted by assessing linear relationships between metabolic concentrations and subject classification defined according to high and low levels in HOMA IR, Matsuda index as well as according to impaired fasting glycemia (Table 6), and taking into account gender. Such analyses confirmed the main associations identified by linear regression analysis for the metabolites with glucose and insulin traits.

TABLE 6
Amino acids and group comparisons at Year 20

| LINEAR REGRESSION, | METABOLITE | | |
|---|---|---|---|
| CORRECTION FOR | CONCENTRATIONAT | | |
| GENDER | AGE 20 | COEFFICIENT | P-VALUE |
| HYDROXYPROLINE | High HOMA-IR Group | 0.1407382 | 0.03261 |
| ORNITHINE | Impaired fasting | 12.1978 | 0.027 |
| | glycemia group | | |
| ALPHAAMINOBUTYRIC | High HOMA-IR Group | −0.509101 | 0.00392 |
| ACID (AABA) | | | |
| ALPHA- | High Matsuda index | 0.523091 | 0.00127 |
| AMINOBUTYRIC ACID | group | | |
| (AABA) | | | |

### Example 4: Multivariate Analysis of Metabolite Concentrations and Clinical Outcomes

Changes in blood amino acid were tested for association with changes in clinical status at year 15 and at year 20. In addition, relationship between the nutrient status at year 15 with the clinical status at year 20 was also assessed.

The Orthogonal Partial Least Squares (OPLS) approach implemented in SIMCA (Umetrics, Sweden) combines both integration and variable selection simultaneously on two data sets (amino acids and clinical variables) in a one-step strategy. It is a multivariate methodology which relates two data matrices X (e.g. amino acids) and Y (clinical variable). PLS goes beyond traditional multiple regression by modelling the structure of both matrices. Unlike traditional multiple regression models, it is not limited to uncorrelated variables. One of the many advantages of PLS is that it can handle many noisy, collinear (correlated) and missing variables, and can also simultaneously model several response variables Y.

The analysis assumed a one predictive component and 2 orthogonal components model was adopted for visualization and interpretation. The results are reported in Tables 7, 8 and 9 as per the coefficient element values for fasting glucose, HOMA-IR and fasting insulin, respectively.

For Fasting glucose, the analysis shows that the combination in the 4 amino acid concentrations at year 15 and 20 contributes to explain glucose level at year 20, with hydroxyproline being the most important metabolite.

Furthermore, since hydroxyproline is produced by hydroxylation of the amino acid proline. Hydroxyproline content is well known marker of collagen catabolism, especially bone resorption or tissue degradation, including muscle damage. Changes in hydroxyproline to proline ratio were positively associated with fasting glucose changes. Specifically, higher conversion of proline to hydroxyproline, yielded a greater fasting glucose.

TABLE 7
Coefficient values for association between metabolites
and fasting glucose obtained by multivariate analysis

| | OPLS DERIVED |
|---|---|
| | COEFFICIENT VALUES |
| | OF VARIABLES WITH FASTING |
| | GLUCOSE AT YEAR 20 |
| NUTRIENT AT YEAR 20 | |
| ORNITHINE | 0.5 |
| HYDROXYPROLINE | 0.8 |
| ALPHA-AMINOBUTYRIC ACID | 0.2 |
| PROLINE | 0.7 |
| RATIO 4HP/PRO | 0.3 |
| NUTRIENT AT YEAR 15 | |
| ORNITHINE | 0.8 |
| HYDROXYPROLINE | 0.9 |
| ALPHA-AMINOBUTYRIC ACID | 0.4 |
| PROLINE | 0.6 |
| RATIO HP/PRO | 0.4 |

* Ratio HP/Pro: Ratio HYDROXYPROLINE/PROLINE

For HOMA-IR, the analysis shows that the combination in the 4 amino acid concentrations at year 15 and 20 contributes to explain HOMA-IR at year 20, with alpha-aminobutyrate and ornithine being the most important metabolites.

Furthermore, since hydroxyproline is produced by hydroxylation of the amino acid proline. Hydroxyproline content is well known marker of collagen catabolism, especially bone resorption or tissue degradation, including muscle damage. Changes in hydroxyproline to proline ratio were positively associated with HOMA-IR changes. Specifically, higher conversion of proline to hydroxyproline, yielded a greater HOMA-IR.

TABLE 8
Coefficient values for association between
metabolites and HOMA-IR obtained by
multivariate analysis

| | OPLS DERIVED COEFFICIENT |
|---|---|
| | VALUES OF |
| | VARIABLES WITH |
| | HOMA-IR AT YEAR 20 |
| NUTRIENT AT YEAR 20 | |
| ORNITHINE | 0.7 |
| HYDROXYPROLINE | 0.2 |
| ALPHA-AMINOBUTYRIC ACID | −0.5 |
| (AABA) | |
| PROLINE | 0.5 |
| RATIO HP/PRO | 0.1 |
| NUTRIENT AT YEAR 15 | |
| ORNITHINE | 0.5 |
| HYDROXYPROLINE | 0.2 |
| ALPHA-AMINOBUTYRIC ACID | −0.4 |
| (AABA) | |
| PROLINE | −0.4 |
| RATIO HP/PRO | 0.3 |

* Ratio HP/Pro: Ratio HYDROXYPROLINE/PROLINE

For Fasting insulin, the analysis shows that the combination in the 4 amino acid concentrations at year 15 and 20 contributes to explain HOMA-IR at year 20, with alpha-aminobutyric acid and ornithine being the most important metabolites.

Furthermore, since hydroxyproline is produced by hydroxylation of the amino acid proline. Hydroxyproline content is well known marker of collagen catabolism, especially bone resorption or tissue degradation, including muscle damage. Changes in hydroxyproline to proline ratio were positively associated with fasting insulin changes. Specifically, higher conversion of proline to hydroxyproline, yielded a greater fasting insulin.

TABLE 9
Coefficient values for association between metabolites and
fasting insulin obtained by multivariate analysis

| | OPLS DERIVED COEFFICIENT |
|---|---|
| | VALUES OF VARIABLES WITH |
| | FASTING INSULIN AT YEAR 20 |
| NUTRIENT AT YEAR 20 | |
| L-ORNITHINE | 0.6 |
| HYDROXYPROLINE | 10.2 |
| A-AMINOBUTYRIC ACID (AABA) | −0.7 |
| L-PROLINE | 10.3 |
| RATIO 4HP/PRO | 0.1 |
| NUTRIENT AT YEAR 15 | |
| L-ORNITHINE | 0.5 |
| HYDROXYPROLINE | 0.3 |
| A-AMINOBUTYRIC ACID (AABA) | −0.6 |
| L-PROLINE | 0.1 |
| RATIO 4HP/PRO | 0.1 |

* Ratio HP/Pro: Ratio HYDROXYPROLINE/PROLINE

Such observations translate with the fact that (i) greater control of glucose management is achieved in subject showing lower blood concentrations in hydroxyproline, proline, ornithine and alpha-aminobutyric acid, and lower conversion of proline to hydroxyproline (i.e. tissue degradation and muscle damage) after an overnight fasting period, and (ii) greater preservation of insulin sensitivity in subjects having lower blood concentration in hydroxyproline, proline, ornithine, lower conversion of proline to hydroxyproline after an overnight fasting period. Subjects unable to achieved glucose control and insulin sensitivity shows a great mobilisation in these amino acids during fasting state that is achieved through enhanced catabolism, and thus these subjects have greater requirements in hydroxyproline, proline, ornithine to maintain body functions including tissue and muscle functions in childhood and adulthood that could be achieved through diet intake rather than unbalanced catabolic metabolism.

### CITED REFERENCES

Arner, P., Andersson, D. P., Backdahl, J., Dahlman, I., and Ryden, M. (2018). Weight Gain and Impaired Glucose Metabolism in Women Are Predicted by Inefficient Subcutaneous Fat Cell Lipolysis. Cell Metab. doi: 10.1016/j. cmet.2018.05.004.
Bates, D., Maechler, M., Bolker, B., and Walker, S. (2015). Fitting Linear Mixed-Effects Models Using lme4. Journal of Statistical Software 67, 1-48. doi: 10.18637/jss.v067.i01.
Hosking, J., Metcalf, B. S., Jeffery, A. N., Streeter, A. J., Voss, L. D., and Wilkin, T. J. (2014). Divergence between HbA1c and fasting glucose through childhood: implications for diagnosis of impaired fasting glucose (Early Bird 52). Pediatr Diabetes 15, 214-219.
Hosking, J., Metcalf, B. S., Jeffery, A. N., Voss, L. D., and Wilkin, T. J. (2011). Little impact of resting energy expenditure on childhood weight and body composition: a longitudinal study (EarlyBird 47). Nutr Res 31, 9-13. doi: 10.1016/j. nutres.2010.12.008.
Jeffery, A. N., Metcalf, B. S., Hosking, J., Streeter, A. J., Voss, L. D., and Wilkin, T. J. (2012). Age before stage: insulin resistance rises before the onset of puberty: a 9-year longitudinal study (EarlyBird 26). Diabetes Care 35, 536-541. doi: 10.2337/dc11-1281.
Jeffery, S. C., Hosking, J., Jeffery, A. N., Murphy, M. J., Voss, L. D., Wilkin, T. J., and Pinkney, J. (2018). Insulin resistance is higher in prepubertal girls but switches to become higher in boys at age 16: A Cohort Study (EarlyBird 57). Pediatr Diabetes 19, 223-230. doi: 10.1111/pedi.12571.
Kuznetsova, A., Brockhoff, P. B., and Christensen, R. H. B. (2016). lmerTest: Tests in Linear Mixed Effects Models. R package version 2.0-30. https://CRAN.R-project.org/package=lmerTest.
Marcovecchio, M. L., and Chiarelli, F. (2013). Obesity and growth during childhood and puberty. World Rev Nutr Diet 106, 135-141. doi: 10.1159/000342545.
Pinkney, J., Streeter, A., Hosking, J., Mostazir, M., Jeffery, A., and Wilkin, T. (2014). Adiposity, chronic inflammation, and the prepubertal decline of sex hormone binding globulin in children: evidence for associations with the timing of puberty (Earlybird 58). J Clin Endocrinol Metab 99, 3224-3232. doi: 10.1210/jc.2013-3902.
Sandhu, J., Ben-Shlomo, Y., Cole, T. J., Holly, J., and Davey Smith, G. (2006). The impact of childhood body mass index on timing of puberty, adult stature and obesity: a follow-up study based on adolescent anthropometry recorded at Christ's Hospital (1936-1964). Int J Obes (Loud) 30, 14-22. doi: 10.1038/sj.ijo.0803156.

1. A method for glucose management comprising administering to a subject in need of same a nutritional composition comprising one or more amino acids selected from the group consisting of: hydroxyproline, proline, ornithine and alpha-aminobutyric acid and mixtures thereof.

2. A method according to claim 1 wherein said nutritional composition contains at least hydroxyproline.

3. A method according to claim 1 wherein said nutritional composition contains at least hydroxyproline and proline.

4. A method according to claim 1 wherein said nutritional composition contains at least hydroxyproline, proline, and ornithine.

5. A method according to claim 1 wherein said nutritional composition contains at least hydroxyproline, proline, ornithine and alpha-aminobutyric acid.

6. A method according to claim 1 selected from the group consisting: of a food product, beverage product, a food supplement, an oral nutritional supplement (ONS), a medical food, and combinations thereof.

7. A method according to claim 1 formulated suitable for administration to a child or adolescent.

8. A method according to claim 1 for use in a method of preventing or treating insulin resistance in a child or adolescent.

9. A method according to claim 1 for use in a method of preventing or treating pre-diabetes in a child or adolescent.

10. A method according to claim 1 for administration to a child or adolescent who is determined as being obese.

11. A method according to claim 1 for use in weight loss during a low caloric dietary intervention or maintenance of weight following a low caloric dietary intervention in a child or adolescent.

12. A method according to claim 11, wherein the one or more amino acids are administered simultaneously to an obese subject as a diet product in the context of a low caloric dietary intervention between 600 kcal/day to 1500 kcal/day for up to 12 weeks.

13. A method according to claim 1, wherein the one or more amino acids are administered simultaneously, sequentially or separately to a subject.

14. (canceled)

15. A method of glucose management in a child or adolescent subject in need comprising the steps of:
i) providing the subject a nutritional composition comprising one or more amino acids selected from the group consisting of: hydroxyproline, proline, ornithine and alpha-aminobutyric acid and mixtures thereof; and
ii) administering the nutritional composition to said subject.

16. A method of glucose management in a child or adolescent subject in need according to claim 15 wherein said subject is determined as being insulin resistant or at risk of being insulin resistant.

17. A method of glucose management in a child or adolescent subject according to claim 16 wherein insulin resistant or risk of being insulin resistant is determined according to measurement of the subject for: fasting insulin levels, glucose tolerance test and Matsuda index, Homeostatic Model Assessment (HOMA), Quantitative insulin sensitivity check index (QUICKI), hyperinsulinemic euglycemic clamp or a modified insulin suppression test compared to a reference age and sex cohort.

18. A method of glucose management in a child or adolescent subject in need according to claim 15 wherein said subject is determined as being pre-diabetic or at risk of being pre-diabetic.

19. A method of glucose management in a child or adolescent subject according to claim 18 wherein said condition of pre-diabetic or risk of being pre-diabetic is determined according to measurement of the subject using a Impaired glucose tolerance (IGT) test and/or Impaired fasting glucose (IFG) test compared to a reference age and sex cohort.

20. A method of glucose management in a child or adolescent subject in need according to claim 15 wherein said subject is determined as being obese by calculation of BMI of the subject compared to a reference age and sex cohort.
